(19) United States
(12) Patent Application Publication
(10) Pub. No.: US 2007/0078294 A1
(43) Pub. Date: Apr. 5, 2007

(54) DYNAMIC RELAXATION AND MOTIVATIONAL AGENT

(76) Inventors: Yogendra Jain, Wellesley, MA (US); Preeti Jain, Wellesley, MA (US)

Correspondence Address: Yogendra Jain, 10 South Woodside Avenue, Wellesley, MA, 02482, US

(21) Appl. No.: 11/470,191

(22) Filed: Sep. 5, 2006

Related U.S. Application Data
(60) Provisional application No. 60/713,692, filed on Sep. 3, 2005.

(51) Int. Cl. A61M 21/00

(52) U.S. Cl. 600/28

(57) ABSTRACT

The present invention provides a Dynamic Relaxation and Motivational Agent (“DRMA”) capable of interacting with a user in real-time and in multi-modes (audio, video, visual, tactile, etc.). Based on user preferences (pre-configured), current request, and the mood of the user, the DRAMA is able to dynamically generate a customized relaxation experience. During the relaxation experience, the user can be motivated and can easily learn and more receptive to suggestions. The DRMA Agent functions as a guide capable of reacting to various user inputs including tactile, physical key/touch screen inputs, voice, breathing noise, silence, background noise, etc.

### CROSS-REFERENCE TO RELATED APPLICATIONS

This application claims priority to U.S. Provisional Application Ser. No. 60/713,692, filed Sep. 3, 2005, which is incorporated herein in its entirety.

### FIELD OF THE INVENTION

The present invention relates to the field of spiritual and physical relaxation through the use of an interactive agent. More particularly, the present invention relates to a system and method for providing an interactive agent for spiritual and physical relaxation which is adaptable to a plurality of users and situations.

### BACKGROUND OF THE INVENTION

There are currently many services and devices available in the marketplace to assist people in various relaxation techniques. For example, there are many audio cassettes and compact discs available which have pre-recorded relaxation routines available. However, these recordings are limited in that they will always contain the same material. The content is not dynamic.

Additionally, a person can also interact with live guide in order to be, instructed in relaxation techniques. While this may have many benefits over recorded relaxation routines (e.g., the content is dynamic and more personal), these solutions can be costly because they require the attention of a single live person for a long period of time. Additionally, the guide has limited knowledge, may not be consistent, may not speak the same language, cannot be at your home when you want to relax, and can be quite expensive. Interacting with the guide over the telephone or net is limiting and that experience and quality of interaction is not possible.

Therefore, there clearly exists a need for a guide that can direct and focus users attention using the appropriate background music, words of wisdom, spiritual words, religious hymns and discourse, prayers, relevant happenings and events, user specific information, and reference past relaxation history, etc. Once the mind and body are relaxed, a user can be easily motivated and ready to listen, learn, and interact.

### SUMMARY OF THE INVENTION

The present invention provides a Dynamic Relaxation and Motivational Agent (“DRMA”) capable of interacting with a user in real-time and in multi-modes (audio, video, visual, tactile, etc.). Based on user preferences (pre-configured), current request, and the mood of the user, the DRMA is able to dynamically generate a customized relaxation experience. During the relaxation experience, the user can be motivated and can easily learn and more receptive to suggestions. The DRMA Agent functions as a guide capable of reacting to various user inputs including tactile, physical key/touch screen inputs, voice, breathing noise, silence, background noise, etc.

The DRMA can be a software application or a physical device like a remote control, or a telephone, video conferencing, PC, music, MP3 like, home gateway, or some combination of these. Additionally, the DRMA can be integrated with pre-existing personified assistants such as is described in co-pending U.S. patent application Ser. No. 11/457,512 filed Jul. 14, 2006.

In home/rooms/devices that have integrated controls, the DRMA can link into this and provide a holistic coordinated control of all of these parameters based on the relaxation program being utilized. For example, the lights are slowly dimmed as the user goes in deep relaxation and lit towards the end of the session. The color of the DRMA device may also change as the user goes in deeper and deeper meditation as determined by bio measurements made by the device.

A user can activate the DRMA simply by speaking to it by a chosen name or through any other detectable action, such as waving a hand in front of the device. Since the device is capable of recognizing users utilizing biometric techniques, the user's preferences can be instantly loaded. The user will then be greeted and asked what function is desired.

During operation, the DRMA interacts with the user according to the predefined settings of the program being utilized. For example, if a user selects a meditation program, the program may first dim the lights in the room and ask the user to start taking deep breaths. The program could then also initiate other meditation techniques such as playing soft and relaxing music or releasing pleasant fragrances through an integrated device. The following are just a few applications for DRMA:
The DRMA system can download new content for mental exercises for users who are at risk of, or have been diagnosed, or have Alzheimer's disease. The DRMA system can ask them to do certain metal exercise, ask those questions; do short term memory games, math games, etc.
Physical Therapy—for users who need guidance in physical therapy, the therapist can recommend or customize a certain interactive exercise and the DRMA system can be the guide, trainer, and monitor user's progress.
Depression—users can get a motivational and psychotherapy reminders as what to do and the device can interact with users with the therapist voice. The Therapist can record on DRMA what the users needs to hear and that can get transmitted to user's device and become part of the relaxation experience.
Learning—The DRMA system can be used for learning new language, new content, game, etc.
Diary—The DRMA system can interact with the users and take a log or personal diary and record it, store it at some appropriately hosted or local storage with appropriate markers such as keep this secure for next 20 years or release this to TBD person, etc.

Once the DRMA has finished its program, it may interact with the user to determine the effectiveness of the program. Based upon the results, the DRMA can use artificial intelligence to modify future sessions of the program with he data gathered. Additionally, this information could be provided in a confidential manner to the author of the DRMA software utilized so that future versions would better suit user needs.

The DRMA device is also expandable in that different programs could be downloaded and installed from a central DRMA server. These programs could be free, based on a subscription plan, or made available at a set price. Additionally, the DRMA server could be utilized to update the DRMA software as newer versions are made available.

### BRIEF DESCRIPTION OF THE DRAWINGS

The above described features and advantages of the present invention will be more fully appreciated with reference to the detailed description and appended figures in which:

FIG. 1 depicts a network diagram of a DRMA system in accordance with a preferred embodiment of the present invention.

FIG. 1 depicts a remote control utilized to interact with the DRMA system.

### DETAILED DESCRIPTION OF THE INVENTION

The present invention provides a Dynamic Relaxation and Motivational Agent (“DRMA”) capable of interacting with a user in real-time and in multi-modes (audio, video, visual, tactile, etc.). Based on user preferences (pre-configured), current request, and the mood of the user, the DRMA is able to dynamically generate a customized relaxation experience. During the relaxation experience, the user can be motivated and can easily learn and more receptive to suggestions. The DRMA Agent functions as a guide capable of reacting to various user inputs including tactile, physical key/touch screen inputs, voice, breathing noise, silence, background noise, etc.

The following is a sample of a guided mediation session. Based on the length of the meditation/relaxation request, the length of each segment can be varied and the subsequent content can be varied based on previous content that was played out.

10 Minute Guided Meditation Example of Relaxation and Guidance

| | | What DRMA | Optional |
|---|---|---|---|
| | What Users does | Says | DRMA Text |
| Start - Breathing | Close your eyes, keep your back | first we'll | When inhaling, |
| | straight), relax, | relax the body, then | stomach should come |
| | Take three deep breaths: | relax the mind, let the | out (because your |
| | Inhale, 2, 3, 4 - Hold, 2, 3, 4, | full energy flow | diaphragm push down |
| | Exhale 2, 3, 4, 5, 6 | through your spine | and your lung capacity |
| | Inhale, 2, 3, 4 - Hold, 2, 3, 4, | Take a deep | expands) |
| | Exhale 2, 3, 4, 5, 6 | breath | |
| | Inhale, 2, 3, 4 - Hold, 2, 3, 4, | Make a nice | |
| | Exhale 2, 3, 4, 5, 6 | strong sound | |
| | | Let all the | |
| | | negativities out . . . | |
| Body Relaxation | Toes: Move your toes around; | Let's relax the | Keep your eyes |
| | stretch your leg muscles, and your thigh | body . . . release some | closed, back straight . . . |
| | muscles, | of the tensions in the | Do it gently . . . |
| | Stretch your figures out wide, | muscles . . | relax, think about what |
| | and bring them in., rotate your wrist in | | muscles you are |
| | one direction, and in another direction | | relaxing, feel the |
| | Raise your shoulders to your | | tension leaving |
| | ears and down . . . (3x), bring your chin to | | |
| | your chest, and head all the way back | | |
| | slowly . . . feel the stretch; | | |
| | Your right ears touch your right | Relax the | Many muscles |
| | shoulder . . . head back, left ears touch | facial muscles . . . | here . . . need to relax it: |
| | your left shoulder, and chin to your | | |
| | chest, (do this 3x) and reverse direction; | | |
| | Open your mouth wide(2x) | | |
| | close, once more, close, raise your | | |
| | eyebrows and close your eyes tight . . . | | |
| | Take a deep breath . . . relax . . . | | |
| Mind Relax | Keep your eyes closed; observe | Now that the | Mind is like a |
| | your breathing . . . and now slowly observe | body is relaxed, we'll | monkey running |
| | your mind . . . see the thoughts . . . observer | relax the mind . . . | around in a cage . . . |
| | the thoughts . . . | Keep your | moves from one |
| | Gently escort your thought out . . . | eyes closed . . . | thought to another . . . |
| | Don't get upset at yourself . . . | | Like a stormy |
| | | | ocean . . . many thoughts |
| | | | rise . . . and as you go |
| | | | deeper and deeper in |
| | | | meditation, the ripples |
| | | | become smaller and |
| | | | smaller . . . |
| Meditate | Topic 1: Body/Soul - Meditate | Now that the | Topic 2: Out of |
| | on I'm the body and the Soul; The Soul | mind is relaxed, we | Body experience |
| | is real me and is eternal and divine - full | will go deeper in our | observe |
| | of karmic bondages . . . and as I'm | meditation . . . | yourself sitting in this |
| | meditating, I'm getting stronger and | | posture; see from the |
| | many of the Karmic bondages are | | top of the room, school, |
| | getting weaker . . . like bonds are getting | | town, state, US, world . . . |
| | weaker . . . | | see the universe, you |
| | They cannot influence me as my | | are free; eternal, you |
| | will power is getting stronger . . . | | have infinite |
| | | | perception, power, etc. |
| | | | Topic 3: |
| | | | Sameru Parvat |
| | | | Meditating on |
| | | | top of the mountain |
| Silence | Meditate on the subject . . . go deep | Keep your | |
| | on the subject . . . | back straight, keep | |
| | | you mind focused . . . | |
| End Session | (in the mind) Om Namo Arhit . . . | Now we'll | Think of the |
| | ..Namo Loya Savshaunam | end the session with | meaning of Namokar |
| | (on our lips) Om Namo. . . . .Namo | three times Namokar | Mantra when changing |
| | Loya. . . . (all together) Om Namo . . . | Mantra . . . once in the | (Bow to the |
| | .Namo Loya . . . | mind; once on the | Arihants - Siddhas - |
| | Inhale - (stomach out) say | lips; and once | Acharya - Upadhyas, |
| | Oooooooooooommm while exhaling | together | and all Sadhus) |
| | Inhale - Oooooooooooommmm | | |
| | Inhale - Oooooooooooommmm | | |
| | You may ask how the meditation | | |
| | was . . . | | |

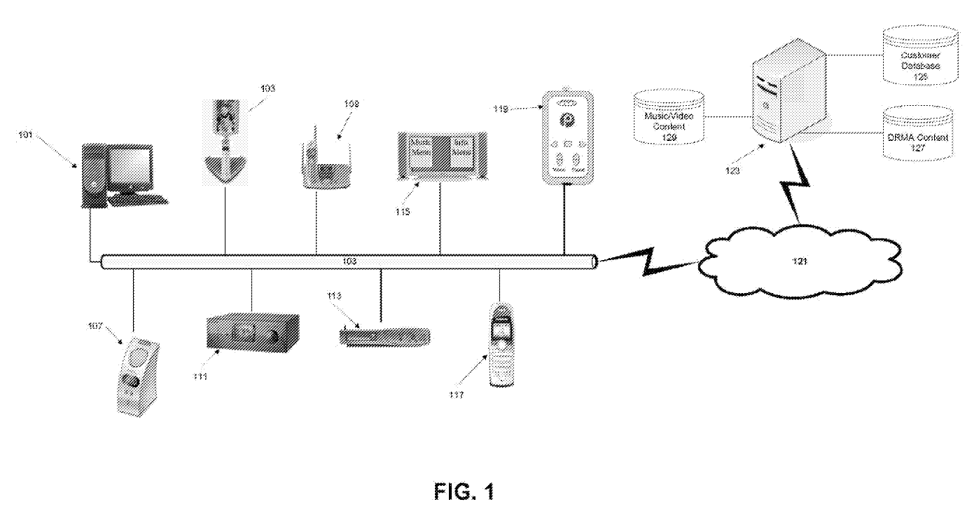

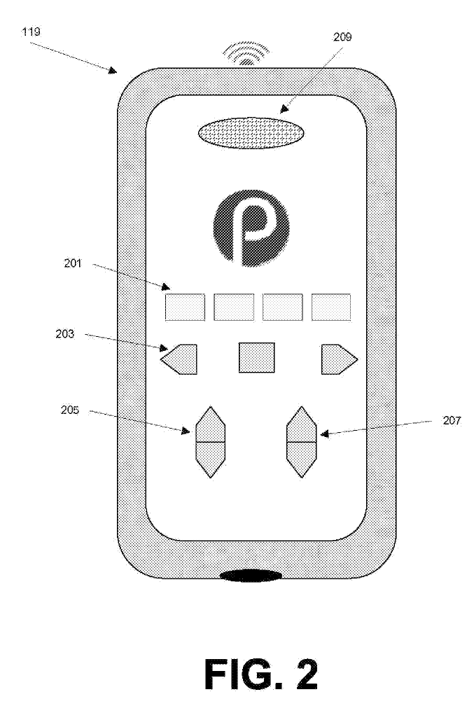

Referring first to FIG. 1, shown is a network diagram which illustrates a DRMA system in a typical home or office. Personal computer (“PC”) 101 hosts the DRMA software. Through a telephone cable, a plug-in USB card, or via broadband, PC 101 is able to access, both live content and stored/cached content. Alternatively, the DRMA software can be located in a custom DRMA system that has the sole purpose of hosting the DRMA software.

PC 101 is connected to various electronic devices within the home through network 103. Network 103 may be an Ethernet network, a wireless network, Bluetooth, etc, or any combination thereof.

PC 101 also stores the user preferences for all users of the system. User preferences stored on the device may include type of relaxation (breathing, muscles/body, spiritual, mind management, etc.) desire, background music preferences, language settings, religion (Christian, Buddhist, etc.), subject of meditation (environment, compassion, forgiveness), imagery, type of motivational guidance, ethnic background. This information may either be loaded into the device by the user or the system may gather this information over time as different programs are selected. Relaxation content may include—inspirational messages; word of the day; sutra of the day; religion of the day; scholar of the day location of the day; meditation of the day; live content download; back lectures listening option on various; subjects by various munis/scholars, etc.

Some configuration may also happen in the background through the use of a live operator while a user is interacting verbally with the DRMA system and the user is not told that there is a live operator in the background that is helping in the customization and configuration. The live operator background mode can be invoked in real-time or par or all of the user session can be recorded and customized later (when an operator of the specialty is available. For example if a users is invoking a configuration for “Church of Jesus Christ of Latter-day Saints” and that operator is not available, user interaction can be recorded and then operator at some later time can do the customization. To guard privacy, the operator is only presented with the data and not with patient's name and other private information.

Through network 103, PC 101 is also connected to Internet Appliance 105. Internet appliance 105 has various I/O connectivity for both hosting different DRMA functions and for interacting with the users of the DRMA system.

A custom DRMA device 107 (such as CCLock/VocalValet) may also be connected to network 103. It has the shape, look, feel of relaxation/meditation/spiritual guide. Custom DRMA device 107 can also contain a hard disk which stores different DRMA content. It can even take a shape of a religious or spiritual guru statue.

Custom DRMA device 107 may also come in a variety of configurations.
Microphone/Speaker/Small Configuration—Custom DRMA device 107 may have a single or multiple microphones to pick up the sound from 360 degrees around the device. A computer processor may be in the device or can be located somewhere on network 103, The device application can be co-hosted on PC 101 or another home device and depending on the desired us of the device.
Human Size “walk in device ”—Custom DRMA device 107 DRMA can be integrated with a physical enclosure which may circular or square in size and able to fit one person in. Another option is that the chamber can fit one person in a sitting posture. In this configuration, custom DRMA device 107 can be soundproof or a closed room containing speakers, microphones, displays, etc.

Telephone 109 allows a user to active the DRMA software by pressing ** or some pre-defined key and talking into it to get. In this manner, even the telephone can function as a remote control for the DRMA system.

Music system 111 may be utilized for playing the DRMA content. Music system 111 may be a home music system, an MP3 Player, or any like device. Additionally, a media player 113 can be remotely positioned in different rooms for playing DRMA content.

DRMA content can also be played directly on television (“TV”) 115. The content can be displayed on screen or a custom device can be connected connect to TV 115 to add text/prayers, images, and sound.

Mobile telephone 117 (Wifi/Skype phones) can also play DRMA content and/or redirect the content to another I/O device located on network 103. Additionally a custom remote control 119 specific for DRMA can be utilized. Remote control 119 has keys for Meditation Relaxation, Prayers, Hymns, Discourse, Motivational, and another set of keys for start, stop, play, pause, and on/off keys. It can connect to PC 101 via local IR remote.

Network 101 is also connected to remote DRMA server 123 via Internet 121. DRMA server 123 stores authored material for downloading. The authored material can also be accessed via different compatible sites. DRMA 123 server contains customer database 125, DRMA content database 127, and music/video database 129. Customer database 125 is preferably an LDAP database which stores user preferences and information about the users of different DRMA devices. Customer database 125 may also store user purchasing information to be utilized if a user devices to purchase all additional motivational/relaxation program through DRMA server 123.

DRMA content database 127 stores additional DRMA programs which can be downloaded by users. Music/video database 129 stores additional multimedia content which may be utilized in conjunction with different DRMA programs. All DRMA based and usable content is indexed and tagged. All aspects of contents are defined such as source, author, Digital Rights, quality, frequency of use by others, popularity, etc.).

Referring next to FIG. 2, shown is a schematic diagram of remote control 119 depicted in FIG. 1. Remote control 119 contains content selection keys 201, control keys 203, time selection keys 205, volume keys 207, and microphone 209. Content selection keys 201 may be labeled Meditation, Relaxation, Prayers, Hymns, Discourse, Motivational, etc. Control keys 203 allow a user to start, stop, play, and pause. Time selection keys 205 allow for a user to vary the length of the program being utilized (e.g., 1 min., 5 min., 10 mill., 30 min.). Similarly, volume keys 207 allow a user to control the volume of the DRMA program. Microphone 209 allows a user to interact with PC 101 through voice commands if the user does not, wish to use the buttons on the remote control. Remote control 119 may even contain a speaker (not shown) so that DRMA content can be played directly through the remote.

Remote control 119 links to PC 101 or custom DRMA device 107 through an infrared signal, a radio link, or other wireless form of communication. Remote control 119 is preferably a lightweight handheld device which is battery operated. The Face plate can easily be changed on the remote control so that it can be easily modified for different languages and specific “channels” (a particular minister/or a priest).

Referring back to FIG. 1, various aspects of the DRMA system will now be explained in more detail:

DRMA Color/Wakeup/Control

The DRMA can be woken up or activated by one or more of these methods: by saying its name (example: “my guru” or another name which the user gives or is already given—DRMA is always listing for its name, user waving his hand near the DRMA device (e.g., custom DRMA device 105); user touches custom DRMA device 105.

The device can periodically wake up based on user setting or mood and ask the user if they would like to have a relaxation session. When the user invokes DRMA or DRMA wakes up, the system color is changed.

At times, users may wish to halt or accelerate the session. DRMA capabilities include:
Stop, start, pause, restart, go back, etc.
Repeat last 30 seconds; go back 5 minutes; fast forward
Color Mood setting—Depending on the type of relaxation; the device can set appropriate color display and settings.

Custom DRMA device 105 can change color based on the level of device application usage. For example, the color may turn dark if the use is none to minimal and turn to bright blue the more use there is. The color change can be part of the device color/lights components or control room or external lights. If there is too much background noise, the colors may flicker to indicate disturbance in the mind and as the noise goes away the noise color indicator changes to a smooth color;

DRMA Mode—Mimicking the User

The DRMA system can also operate in a mode where it is not only doing the guiding but also is participating in the meditation with the users. This makes the users feel just as important in leading the session.

Additionally, the DRMA system may get users to give guided meditation as the users learn more and more. The DRMA system may also make breathing noises as it is doing the relaxation (if users are doing breathing exercises)

The DRMA system can play background music and encourage the users to say the prayers or sing a particular devotional song or poem or prayers, etc. The DRMA system may jointly chat with users during appropriate sessions (like a prayer) if it knows it. If it does not know the prayer, it may convert users' speecliprayer in text and look for that prayer on DRMA server 123. A portion of the prayer that the users is saying can be sent to DRMA server 123 and a live domain specific operator at the portal can recognize it and download the recognized prayer for display on the screen or offer background music.

Error Handling

The DRMA system handles errors by taking some of the blame on itself and sharing the blame with user and other environmental factors. Some errors may be that the DRMA system may not understand user spoken commands and blames it on the background noise or too much sound reflection in the room; attributes the problem to internet being too slow or not accessible or down; attributes the problem to user speaking too softly or fast. At times, the DRMA takes the blame and apologizes.

The DRIA system may get another agent to take and give blame in case of major errors with DRMA sub-systems instead of the “relaxation” guide. The “relaxation” guide may or may not hint towards the error. For example if the internet goes down and fresh content cannot be served, the DRMA system may get the device default agent to say “John—this is your device (name)—my apologies to you and your “relaxation guide ” as the internet is down or some type of error has taken place. Such intervention allows “relaxation guide” to focus on the relaxation application.

Prayers/Chanting/Group Sessions

The DRMA system can lead (or as directed by a priest or a monk or a group leader) group chanting or prayers.
In chant mode it can sing the song or prayers in low volume and users can follow it.
It can provide background music for particular prayers and users can chant it.
If a display exists, it can display the text, lights, and appropriate video;
DRMA can host a discussion group in a chat room type forum for a group of people.

Multiple DRMAs at local or remote locations can synchronize during a relaxation program. A central hosted server or one appropriately selected among the many logged in can distribute/stream to all the DRMAs. DRMA can play that out or appropriately process it.

When multiple DRMAs are communicating, several communication modes are possible:
User hears the master DRMA only and user's log in information maybe hidden.
The local DRMA may only transmit command to users (coming in from master) but outgoing noise/background noise on each of the remote DRMAs is not transmitted (like audio conferencing) to all users.
In a Relaxation/Conferencing mode the users may wish to interact pre- or post or during the session. The session may start like an audio conferencing mode and then DRMA takes over and guides the users on the next steps.

Sleeping:

The DRMA system can be as an aid for sleeping. If a user is tired, the DRMA system will make it easier for people to fall asleep by executing a ‘sleeping’ guided relaxation.

The DRMA system can be configured to guide people to sleep by initially interacting with the user (or users default parameter) and then determine what is the best way to guide someone to sleep. The system may measure appropriate physiological (Oxygen level, etc.) and visual parameters (such as eye) and determine the level of sleep. Based on this, users may start a customized or previously available application.

If the users fall asleep and the DRMA system can detect no response from the user, it may play some quite music and whisper to the user to see if he/she is awake. For example the DRMA system may ask ‘Are you sleeping?’ in a soft voice.

If the session is in the late evening, then the DRMA system will not wake up the user. If it is during the daytime, then based on previous sessions and user configurations, wake up would commence in 5, 10, 15 minutes as appropriate.

If the DRMA system has accessibility, and the user has given permission in the configuration file, DRMA can turn off the telephone ringer and divert calls to DRMA and/or voice mail and when the user finishes the relaxation session, give the messages to the users.

DRMA Physiological Data Collection

As part of enhancing the Relaxation experience, the DRMA system may integrate with the following types and classes of devices. Specific integration can be via a wired connection, wireless, periodic update, etc. Sample physiological devices/measurements include the followings
Oxygen level measurement, EKG, EEG, blood pressure, heart rate, eye scan, posture, etc. Such measurements can be used by the DRMA system to give users multi-modal (voice, visual, tactile) auto/DRMA agent feedback.
Multiple physical devices can pass the data through an interface such as USB, RS232, etc, or by using any of the interfaces (speech, key board, home telephone, etc).

Such data can be recorded for comparison later and shared with the user as to the changes. The DRMA system can send a plot or analysis of the change as well.

The DRMA system can also share, with appropriate approval from the user, the plot, analysis, and raw data with user's physician, health care worker, family member, friend, etc.

The DRMA system will keep such medical data secure and follow the HIPAA requirements.

The DRMA system can also input user medical information (example: heart disease, cholesterol level, diabetes, depression, etc.) and focus some of the relaxation customized to such medical problems.

Background Functions Performed by DRMA:

The DRMA system may report various usage metrics based on what types of programs users have accepted or selected.

The DRMA system also gives users summaries of the “amount of relaxation” done “this past week” This summary can be sent via email, verbal feedback to the user, display, or any combination of these or other type of available display in the home.

The DRMA system may also embed local weather/any critical news/holidays/religious festivals in the “Relaxation” sequence (Example: Even thought the weather today is rainy and cold, let's focus on the warm and cozy home . . . ).

The network operator or the content developer can check the progress of the sessions which have been downloaded and are being actively used and dynamically tune the message to a group of users. For example, a request for donation can be made after the DRMA session has ended or even interruption in the middle of the session if appropriate (such as hurricane warning).

Remote Use

The DRMA system can be portable such that it can be used/installed in hotel, take it on vacation. Preferences settings and personal information can be stored locally or in a hosted secure network such that users can access it when they are traveling.

Hotels, resorts, yoga centers, local fitness center/gym, church/temple, or just a relaxation group can provide additional content for free or for some fee depending on the application. When the DRMA system is turned on, it may directly connect to a hosting server, identify itself, and same experience that the users had in their home can be had at remote locations.

1. A dynamic relaxation and motivational agent comprising:
at least one personal computer;
at least one set of speakers;
at least one microphone; and
a network which interconnects said personal computer, said speakers, and said microphones; and
relaxation software located on said computer which utilizes said speakers and said microphone to lead a user through an interactive relaxation sequence.
